(19) United States
(12) Patent Application Publication
(10) Pub. No.: US 2005/0042173 A1
(43) Pub. Date: Feb. 24, 2005

(54) Micronized film-forming powder comprising an active substance

(76) Inventors: Jerome Besse, Listrac Medoc (FR); Laurence Besse, Listrac Medoc (FR); Philippe Cornu, Rueil-Malmaison (FR); Brigitte Taravella, Paris (FR)

Correspondence Address: JACOBSON HOLMAN PLLC, 400 SEVENTH STREET N.W., SUITE 600, WASHINGTON, DC, 20004, US

(21) Appl. No.: 10/496,094

(22) PCT Filed: Nov. 21, 2002
(86) PCT No.: PCT/FR02/04000

(30) Foreign Application Priority Data
Nov. 21, 2001 (FR) 01/15069

(51) Int. Cl. A61L 9/04; A61K 9/14

(52) U.S. Cl. 424/46; 424/489

(57) ABSTRACT

The present invention relates to a micronized film-forming powder having a particle size of at most 100 μm and comprising a combination of at least one active substance, at least one bioadhesive agent, and at least a plasticizer.

The present invention relates to a micronized film-forming powder, pharmaceutical, cosmetic or nutraceutical compositions containing this powder, as well as to methods for their manufacture and their uses.

The micronized film-forming powder according to the present invention possesses the specific feature of forming a film in situ at the time of its application on a moist or hydrated support. It can be applied to the dermis and/or to a mucous membrane.

Due to the fact that this powder forms a film on the dermis or on the mucous membrane during its application, it allows the sustained prolonged release of the active substance(s) which it contains. This sustained release can occur in several ways. For example, linearly or with a “burst effect” (immediate release of part of the active substance, followed by a sustained release), also called “bimodal release profile” or “rapid and sustained release effect”.

A decisive advantage of this galenic form consists in the fact that the film erodes with time so as to leave no residue.

Fluid compositions capable of forming films in situ during their application are already known. Thus, patents U.S. Pat. No. 5,081,157 and U.S. Pat. No. 5,081,158, and patent applications WO 96/30000, WO 97/31621, WO 00/10540, WO 00/38658, WO 01/13955 and WO 01/43722 describe film-forming compositions for transdermal and/or transmucosal application. These compositions may be in the form of a solution, a suspension or a gel.

The film-forming compositions already known in the prior art suffer from numerous disadvantages. Among these, there may be mentioned the difficulties of preparation linked to the production of compositions which can then form a homogeneous film, the difficulties of storing these galenic forms because they are often unstable, and the difficulties linked to their administration.

In particular, liquids, just like gels, are difficult to position precisely on the dermis or the mucous membranes, and tend to slide or to move.

Moreover, many muco-adhesive preparations known in the state of the art have such a thickness that they result in a discomfort for the individual which uses them, especially when these preparations are applied on buccal or vaginal mucosa.

Also, many preparations known in the state of the art, including the patch type or wafer type preparations, comprise a support which stays in place after the complete release of the active substance(s), which requires an intervention of the practicien to remove them, particularly after applying to the internal mucosa such as the vaginal mucosa.

The applicant companies have therefore sought to develop a galenic form which can overcome the disadvantages encountered by the earlier formulations.

They have thus succeeded in developing a micronized film-forming powder which has the properties of forming a cohesive continuous film when it is contacted with a hydrated or moist support, for example, with mucous membranes or the previously hydrated skin. The film which is formed after applying the micronized film-forming powder to the hydrated or moist support has good adhesive and cohesion properties.

The film-forming composition in the form of a micronized powder according to the invention, unlike the fluid products of the prior art, does not require the use of any liquid carrier, especially solvents, during the administration of the product. This is quite obviously a decisive advantage for a product for pharmaceutical, cosmetic or nutraceutical use. The micronized powder form also allows a very good stability of the product during storage, greater than that of products in the form of solutions, suspensions or gels.

The micronized film-forming powder according to the present invention therefore has numerous advantages compared with galenic forms known in the prior art.

Accordingly, the present invention relates to a micronized film-forming powder having a particle size of at most 100 μm and comprising the combination of at least one active substance, at least one biocompatible adhesive agent, and at least one plasticizer.

By active substance, it is meant according to the invention any substance having a measurable activity, therapeutical, cosmetic or nutraceutical in nature, towards the human or animal body to which this substance is applied or administered.

By biocompatible adhesive agent, which may also be referred to as “bioadhesive agent”, it is meant according to the invention any substance or any compound which has the property of adhering to a hydrated or moist biological tissue when said substance or said compound is applied to it, such as for example a mucous membrane or the previously hydrated dermis. To be biocompatible, the adhesive agent has to be compatible with an use on the biological tissue, without causing unwanted reactions such as an inflammation of the biological tissue.

By plasticizer, it is meant any substance or any compound capable of improving the mechanical properties of the film formed from micronized film-forming powder according to the invention in order to promote the physical integrity of the film during its formation and to maintain the physical integrity of the film after its formation, notably by promoting the cohesion between the particles initially contained in the micronized film-forming powder.

The micronized film-forming powder according to the invention has the properties of forming a cohesive continuous film in contact with the aqueous medium. When said powder is contacted with a moist or hydrated support, preferably the mucous membranes or the previously hydrated dermis.

The film is formed very quickly, from the first minute after the application of the micronized film-forming powder to the surface of the moist or hydrated support, for example mucous membranes or previously hydrated dermis, as illustrated in the examples.

The particle size of the micronized film-forming powder according to the invention is essential for obtaining a film which is cohesive and continuous throughout the surface of the support on which said powder is applied.

Thus, a continuous and cohesive film having good adhesive properties is obtained on the hydrated or moist support with a micronized film-forming powder as defined herein and having a particle size of at most 50 μm as well as with a micronized film-forming powder having a particle size of at most 20 μm, as described in the examples.

Excellent results were also obtained with a micronized film-forming powder having a particle size close to 10 μm.

Accordingly, also being part of the invention is a micronized film-forming powder having a particle size of at most 10 μm.

The micronized film-forming powder has a particle size of at least 0.01 μm, preferably at least 0.1 μm and most preferably at least 1 μm.

Preferably, a micronized film-forming powder as defined above has a particle size of between 0.01 μm and 100 μm, preferably between 0.1 μm and 70 μm and more preferably between 1 μm and 50 μm.

By “particle size” of a micronized film-forming powder according to the invention, it is meant the mean size of the grains that constitute it. The mean size of the grains can be measured by any conventional technique known per se. Notably, the persons skilled in the art can use a measure with the aid of a laser granulometry device of the Beckman Coulter® or Malvern® type, as described in the examples.

The applicant has noticed that the grain size distribution of the micronized film-forming powder according to the invention follows a narrow Gauss curve, with the particle size value corresponding therefore to the real size of the most part of the particles contained in said powder.

The micronized film-forming powder of the invention conveniently has a residual humidity of between 0.1% and 10%, preferably between 2% and 8%, as measured with a humidity analyser type MA 30 sold by the Sartorius Company and used in accordance with the manufacturer recommendations, as illustrated in the examples. The low relative humidity of the micronized film-forming powder according to the invention allows for a storage time of several months without affecting its particle size features nor its properties of forming a continuous and cohesive film when it is applied to a moist or hydrated support.

Moreover, when the micronized film-forming powder according to the invention is applied to a moist or hydrated support, the continuous and. cohesive film which is formed on the surface of the support has a reduced thickness of between 10 μm and 1 mm, preferably between 50 μm and 400 μm and most preferably between 100 μm and 300 μm.

The low thickness of the continuous and cohesive film produced through the application of the micronized film-forming powder of the invention avoids, or to say the least decreases, the discomfort sensations experienced with certain of the devices known earlier. Furthermore, the low thickness of the film formed in this way, because of the lesser mean distance between the active substance(s) it contains and the target sites of those active substances, for example the target sites located on the surface of a mucous membrane, allows an improved accessibility or bioavailability of the active substances towards their target sites and promotes the release of all of the active substance(s)contained initially in said film.

The adhesiveness of a film formed on a moist or a hydrated support from the micronized film-forming powder according to the invention is illustrated by the fact said film has an adhesiveness index or tack of between 1N and 50N, preferably between 2N and 10N.

In order to measure the adhesiveness index or tack, the persons skilled in the art will conveniently use the so-called “Probe Tack” test performed with a traction device, said test being defined in the ASTM standard n° D 297901 (“Standard Test Method for Pressure-Sensitive Tack of Adhesives using an Inverted Probe Machine”—American Society for Testing and Materials), as illustrated in the examples.

The good properties of adhesion to the support of the film formed from the micronized film-forming powder according to the invention avoid, or to say the least decrease considerably the risks of detaching the film from the support on which said film is formed or the risks of sliding or displacement of said film on the surface of the support, which further reduces the possible loss of active substance molecules which do not reach the intended target sites.

Finally, it was shown according to the invention that the film formed after applying the micronized film-forming powder according to the invention to a hydrated or moist support has a good resistance to liquids, which constitutes a technical feature particularly advantageous considering the surfaces on which said micronized film-forming powder is likely to be mostly applied, namely the mucous membranes and the dermis. The good resistance to liquids of said film allows to characterize it as a semipermeable film. The semipermeable character of the film formed from the micronized film-forming powder of the invention is illustrated by the fact that, while the contact angle of said film decreases with its exposition time to different types of liquids, no complete absorption of these different types of liquids is seen whatever the pH, acid, basic or neutral, of the latter, as shown in the examples.

Generally, by allowing the formation in situ of a film having the above technical features, the micronized film-forming powder according to the invention allows a stabilization and a great effectiveness, including therapeutical, cosmetic an nutraceutical effectiveness, of the final product.

Preferably, the micronized film-forming powder according to the invention comprises, on the basis of the total weight of the composition, from 0.001% to 90% by weight of active substance(s), from 1% to 90% by weight of biocompatible adhesive agent(s) and from 0.1% to 30% by weight of plasticizer(s).

The persons skilled in the art adapt. the proportions of the different components of the micronized film-forming powder in accordance with conventional techniques for preparing galenic formulations such as for example the one described in J. Control release: 61(1999) 175-183; J. Pharm (2000) 271-277, J. Control release.77 (2001) 1-6 and J. Pharm. Pharmacol. 48, 255 (1998), so that the powder might have the physical, mechanical and chemical features defined previously for the film formed from this powder, namely the thickness, adhesiveness index, resistance to liquid and semipermeability features.

For example, for an active substance such as the ioded polyvinylpyrrolidone, which is an active substance having by itself adhesive properties, the micronized film-forming powder wild have a low level of biocompatible adhesive agent.

The micronized film-forming powder according to the invention is characterized in that it may further comprise at least a compound selected from a surfactant, a wetting agent, a binder, a retardant, a penetration enhancer, a bioerodible diluent, a colorant, a flavour, a pH controlling agent or a combination of at least two of these compounds.

The surfactants, wetting agents, binders, retardants, penetration enhancers, other than those already acting as a biocompatible adhesive agent or plasticizer, are added.

The active substances of the micronized film-forming powder according to the invention may be selected from those conventionally used in the following specialities: allergology, anaesthetic/intensive care, cancerology and haematology, cardiology and angiology, contraception and abortion, dermatology, endocrinology, gastroenterohepatology, gynaecology, immunology, infectiology, metabolism and nutrition, neurology/psychiatry, ophthalmology, ear, nose and throat, pneumology, rheumatology, stomatology, toxicology, urology/nephrology, and from analgesics and antispasmodics, anti-inflammatory agents, contrast products used in radiology, haemostatics, and products for treating blood and derivatives. But also selected are all cosmetic and/or nutraceutical substances.

Advantageously, the active substances may be selected from the group consisting of the active substances crossing the skin barrier and reaching the systemic circulation, such as cyproterone acetate, Δ4androstenedione, 3-ketodesogestrel, desogestrel, gestodene, estradiol and its derivatives, norethisterone acetate, progesterone, testosterone, trinitrine, fentanyl, nitroglycerine, nicotine (S(−)-nicotine), scopolamine, clonidine, isosorbide dinitrate, levonorgestrel in combination with ethinylestradiol or with estradiol, androstanolone, alclometasone dipropionate, and combinations thereof.

They may also be selected from the active substances crossing the skin barrier and having a localized action such as: acetazolamide, acyclovir, adapalene, alclomethasone dipropionate, amcinonide, ameleine, bamethan sulphate+escin, betamethasone valerate, betamethasone dipropionate, bufexamac, caffeine, calcipotriol monohydrate, cetrimonium bromide, clobetasol propionate, crilanomer, desonide, dexpanthenol, diclofenac, diflucortolone, valerate, difluprednate, diphenydramine hydrochloride, econazole nitrate, erythromycin, flumetasone pivalate, fluocinolone acetonide, fluocinodine, fluocortolone, fluocortolone hexanoate, fluocortolone pivalate, hydrocortisone, hydrocortisone acetate, ibacitabin, ibuprofen, imiquimod, ketoconazole, ketoprofen, lidocaine, metronidazole, miconazole nitrate, minoxidil, niflumide acid, penciclovir, benzoyl peroxide, piroxam, iodinated povidone, promestriene, pyrazonibutasone, roxithromycin, sulphacetamide, triamconolone, tazarotene, tretinoin and isotretinoin, triclocarban, vidarabine monophosphate and combinations thereof.

They may also be selected from the following active substances: β₃-adrenergic agonist, growth hormone, oxybutinin, buprenorphine, pergolide, estradiol+nestorone, nestorone, 7α-methyl-19-nortesterone, mecamylamine (antagonist of nicotine)+nicotine, salbutamol, selegiline, buspirone, ketotifen, lidocaine, testosterone+estradiol, ketorolac, eptazocine, insulin, α-interferon, prostaglandines, 17β-estradiol+norethindrone acetate, 5-aminolevulinic acid, benzodiazepine alprozolam, diclofenac, fenoprofen, flubiprofen, ketoprofen, methyl phenidate, miconazole, piroxicam, bruprenorphine, alprozolam, dexmedetomidine, prazosin (α-adrenergic antagonist), gestodene+ethinylestradiol, alprostadil, tulobuterol (β-adrenergic agonist), ethinylestradiol+norelgestromin, ketorolac, physostigmine, lidocaine, medindolol (α-adrenergic agonist), rotigotine (D2 dopamine antagonist), ethinylestradiol+norethindrone acetate, thiatolserine, and combinations thereof.

They may also be selected from the active substances known to undergo a liver first pass effect such as:
17 β Estradiol, Molecules undergoing first pass effect (non limiting)
17β Estradiol
Ethynylestradiol
Finasteride
Testosterone
Isotretinoin
Biphosphonates
Nicotine

They may also be selected from the active substances which undergo a gastrointestinal degradation such as:
Omeprazole
Acamprosate
Sodium valmate
Esomeprazole magnesium trihydrate
Sodium diclofenac

They may also be selected from the active substances which have a low bioavailability

The micronized film-forming powder may contain one or more active substances, combined with each other.

For cosmetic applications, the active substance may be chosen from the group comprising emollients, moisturizing agents, vitamins, complexes of fruit amino acids, antioxidant agents and the like.

For nutraceutical applications, the active substance may be chosen from the group comprising vitamins, inorganic salts, brewers' yeast and the like.

According to a preferred embodiment of the powder, according to the invention, the active substances are micronized before being mixed with the other ingredients. It is also possible to mix the nonmicronized active substance with the other ingredients of the powder and then to micronize the final mixture. This promotes the homogeneity of the film and the cohesion and adhesion of the particles on its application support. Moreover, systems for spraying powder are particularly well suited to the spraying of micronized products.

The bioadhesive agent of the micronized film-forming powder according to the invention is advantageously selected from the group consisting of ethyl cellulose, methyl cellulose, carboxymethyl cellulose, carboxymethyl cellulose sodium, hydroxyethyl cellulose, hydroxypropyl cellulose, hydroxypropyl methyl cellulose sodium, polyvinylpyrrolidone, polyvinyl alcohols, polyisobutylene, polyisopropene, xanthan gum, locust bean gum, chitosan, chitosan chloride, polycarboxylates, carbomers such as carbopol, acrylic/methacrylic acid copolymer, acrylic acid/acrylamide copolymer, acrylic acid/methyl methacrylate copolymer, acrylic acid/polyethylene glycol copolymer, polyacrylic acid/butyl acrylate copolymer, HEMA (2-hydroxyethyl methacrylate) copolymerized with Polymeg® (polytetramethylene glycol), Cydot® marketed by 3M (carbopol combined with polyisobutylene), pectin (of low viscosity), polyethylene oxide, methyl vinyl ether/maleic anhydride copolymer, tragacanth, monomethyl ether, monomethacrylate, drum dried waxy maize starch, sodium stearyl fumarate, sodium hyaluronate, guar gum, sodium alginate, starches, dextran and derivatives, and mixtures thereof.

The plasticizer of the micronized film-forming powder according to the invention is conveniently selected from the group consisting of dibutyl phthalate, dibutyl sebacate, acetyltributyl citrate, acetyltriethyl citrate, tributyl citrate, tributylethyl citrate, triacetin, PEG, propylene glycol, glycerol, glycerol monoesters and derivatives, castor oil and derivatives, and mixtures thereof.

The micronized film-forming powder according to the invention may also comprise one or more surfactants, which are preferably nonionic, such as polyoxyethylene sorbitan (fatty acid ester, polyoxyethylene alkyl ether, polyoxyethylene derived from castor oil and derivatives, and mixtures thereof.

If necessary, the micronized film-forming powder may also comprise a wetting agent selected from the group consisting of polyols such as sorbitol, or glycerine, such as PEG and mixtures thereof.

The micronized film-forming powder according to the invention may also comprise a binder selected from the group consisting of acacia, alginic acid, carboxymethyl cellulose sodium, microcrystalline cellulose, dextrins, ethyl cellulose, gelatin, glucose, guar gum, hydroxypropyl methyl cellulose, methyl cellulose, polyethylene oxide, povidone, pregelatinized starch and derivatives, and mixtures thereof.

The micronized film-forming powder according to the invention may also comprise a hydrophilic or nonhydrophilic retardant selected from the group consisting of hydroxypropyl methyl cellulose acetate or succinate, hydroxypropyl methyl cellulose, hydroxypropyl cellulose, ethyl cellulose, hydroxyethyl cellulose, carboxymethyl cellulose sodium, polyvinyl alcohols, hydrocolloids such as: pectins, alginates, guar gum, xanthan gum, gum Arabic, agar, dextrin, carragheenan, polyethylene oxide, carbomers, polymers and copolymers of acrylic acid, of methyl methacrylate, of polyvinyl acetate, of carboxymethyl acetate and mixtures thereof.

The micronized film-forming powder according to the invention may also comprise a bioerodible diluent selected from the group consisting of calcium or sodium carbonate or bicarbonate, sucrose, mannitol, xylitol, sorbitol, lactose, cellulose or microcrystalline cellulose powder, starch and its derivatives, dibasic calcium phosphate, tribasic calcium phosphate, calcium sulphate, dextrates, dextrins, dextrose excipients, fructose, kaolin, lactitol and mixtures thereof.

The micronized film-forming powder according to the invention may also comprise a penetration enhancers which may be selected from the group consisting of aliphatic fatty acid esters such as isopropyl myristate, fatty acids such as oleic acid; alcohols or polyols such as ethanol, propylene glycol and polyethylene glycol; components of essential oils and terpenic derivatives (such as eugenoi, geraniol, nerol, eucaiyptoi, menthol); surfactants; moisturizers such as glycerin, urea; keratolytics such as alpha-hydroxy acids.

The micronized film-forming powder according to the invention may also comprise a colorant selected from the group comprising Curcumino, Lactoflavin (riboflavin), Tartrazin, Quinoline Yellow, Orange-Yellow S, Cochineal carminic acid Azorubine, Amaranth, Cochineal-red A Brillant Blue V, Indigotin (indigo carmine), Chlorophylls, Cupric complexes of chlorophylls and chlorophyllins, Caramel, Black, Brillant BN, Carbo medicinalis vegetalis, Carotenoids, alpha, beta or gamma caroten, Bixin, Norbixin, (rocou Annatto), Capsantein, Capsorubin, Lykopene, Xanthophyils, Flavoxanthine, Lutein, Kryptoxanthine, Rubixanthine, Violoxanthine, Rhodoxanthine, Beet Red, Betanin, Anthocyanins, Calcium carbonate, Titanum bioxide, Iron oxides or hydroxides, Aluminum, Silver, Gold.

The micronized film-forming powder according to the invention may also comprise a flavor selected from the flavors conventionally used in pharmacy.

The micronized film-forming powder according to the invention may also comprise a pH-controlling agent. The pH-controlling agents allow to stabilize or to promote the passage of the active substance(s) through the biological support on which the micronized film-forming powder according to the invention is applied. The pH-controlling agents can control the thus formed film pH between pH 2.0 and pH 9.0.

Preferably, the pH-controlling agent is selected from the group comprising citric acid and its derivatives, phosphoric acid and its derivatives, tartaric acid and its derivatives, bicarbonic acid and its derivatives, or a combination of at least two of the above pH-controlling agents.

The invention also relates to a pharmaceutical, cosmetic or nutraceutical composition comprising the micronized film-forming powder as defined in the present description. This composition may be applied to the dermis or the mucous membranes.

When it is administered by the mucosal route, it may be applied, for example, through the buccal mucosa, the nasal mucosa or the vaginal mucosa.

When the micronized film-forming powder according to the invention is administered by the transdermal route, it will have a systemic effect and/or a local effect according to the nature of the active substance and the other components present in the powder.

Advantageously, the composition according to the invention, comprising the micronized film-forming powder, exists in a pulverizable dry form. This allows easy delivery of a precise dose.

The invention also relates to a process for the preparation of a micronized film-forming powder.

All the processes known to persons skilled in the art may be used in the context of the production of this micronized film-forming powder.

There may be mentioned, as an example of a method for preparing a powder wet or dry granulation, by extrusion, by atomization followed by micronization in order to obtain a micronized powder.

Or according to another embodiment, the active substance is micronized and then mixed with the excipients in powdered form, and the mixture thus obtained is granulated, by wet or dry granulation before a new step of micronization.

In every case, the method for manufacturing the micronized film-forming powder of the invention must include a micronization step of the mixture comprising the active substance, the biocompatible adhesive agent and the plasticizer.

For the micronization, the conventional air jet method is used, for example by using an air jet micronization equipment type ALPINE or JET MILL, in accordance with the manufacturer recommendations.

The preferred parameters for a micronization on a micronizer GALETTE Alpine 200AS are the following:
Injector: 7 to 8 Bars;
Crown: 4 to 6 Bars;
Speed: 25 kg/h.

In a particular test performed by the Applicant, before micronization, the powder had a grain mean size (particle size) of 110 μm. After micronization, the resulting micronized film-forming powder had a particle size of 3 μm.

The micronized film-forming powder according to the invention may be used with or in any device allowing its application on the surface of a moist or hydrated support, such as the mucous membranes or the previously hydrated dermis.

Thus, the invention also relates to any device for applying or dispersing the powder on the surface of a support, suitable for use in cosmetic, pharmacy or nutraceutic.

The invention will be understood more clearly with the aid of figures and the examples described below.

### FIGURES

FIG. 1 illustrates the grain size distribution profile of the micronized film-forming powder prepared in the example 2.

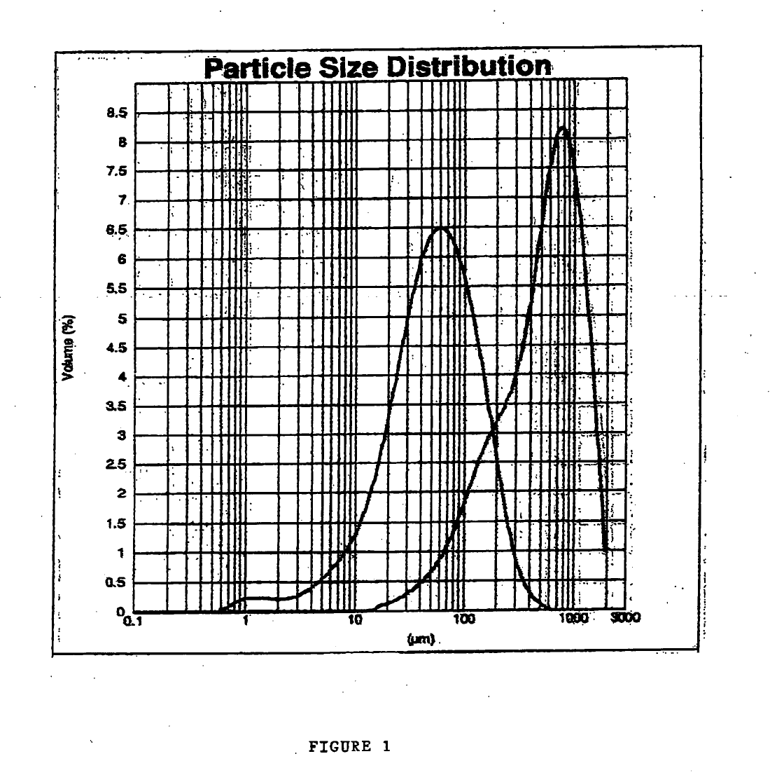

The right curve represents the grain size distribution profile before micronization. The left curve represents the grain size distribution profile after micronization.
In abscissa: Particle size given in μm.
In ordinate: Volume, given as a percentage.

FIG. 2 illustrates the grain size distribution profile of the micronized film-forming powder prepared in the example 3.

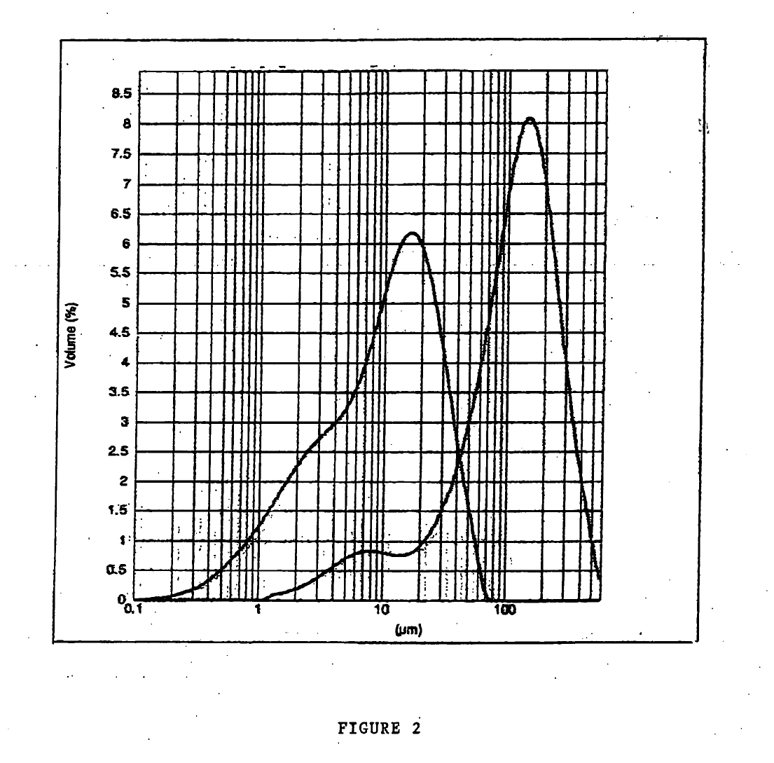

The right curve represents the grain size distribution profile before micronization. The left curve represents the grain size distribution profile after micronization.
In abscissa: Particle size given in μm.
In ordinate: Volume, given as a percentage.

### EXAMPLE 1

### Micronized Film-Forming Powders According to the Invention

Four powders, each having the following composition by weight, are prepared:

TABLE 1

| Components | Quantity in % |
|---|---|
| Buprenorphine | 3 |
| Microcrystalline | 67 |
| cellulose | |
| Kollidon ® VA64 | 10 |
| Ethylcellulose | 20 |

TABLE 2

| Components | Quantity in % |
|---|---|
| 17β-Estradiol | 5 |
| Microcrystalline | 55 |
| cellulose | |
| Kollidon ® VA64 | 10 |
| Ethylcellulose | 20 |
| Dibutylphthalate | 10 |

TABLE 3

| Components | Quantity in % |
|---|---|
| Molsidomine | 7 |
| Eudragit ® RSP0 | 30 |
| Polyvinyl alcohol | 3 |
| Microcrystalline cellulose | 50 |
| Triethyl citrate | 10 |

TABLE 4

| Components | Quantity in % |
|---|---|
| Salbutamol | 5 |
| HPMC Succinate acetate | 30 |
| Carbopol ® 974 PNF | 3 |
| Microcrystalline cellulose | 51 |
| Triethyl citrate | 10 |
| Sodium lauryl sulfate | 1 |

The various components are mixed in a mixer-granulator of the mixer-granulator-vacuum drier type ROTOLAB ZANCHETTA or equivalent until the mixture is homogenized. Next, a wetting solution or suspension is incorporated, with stirring, in order to obtain a wet granulate.

This granulate is then dried under suitable conditions so as to evaporate the granulation solvent. This granulate is then calibrated.

### EXAMPLE 2

### Micronized Film-Forming Powder According to the Invention

A powder having the composition by weight described in detail in the table 5 below is prepared.

TABLE 5

| Components | Quantity in % |
|---|---|
| Buprenorphine | 3 |
| Hydroxypropyl methyl cellulose | 43.65 |
| Carbopol 974P NF | 43.65 |
| Eudragit RSPO | 9.7 |

Manufacturing Process

The various components are mixed in a mixer-granulator of the mixer-granulator-vacuum drier type ROTOLAB ZANCHETTA or equivalent until the mixture is homogenized. Next, a wetting solution or suspension is incorporated, with stirring, in order to obtain a wet granulate.

This granulate is then dried under suitable conditions so as to evaporate the granulation solvent and then calibrated and micronized with a micronizer type GALETTE Alpine 200AS by using the following paramaters:
Injector: 8 Bars;
Crown: 4 Bars;
Speed: 25 kg/h.
   Final Product Control
   Particle Size:

Carried out by using a laser granulometer MASTER SIZER 2000 equipped with a vibrator Scirocco 2000.

Result: mean particle size: before micronization=559.133 μm; after micronization=54.242 μm.

Relative Humidity Level Measurement

Carried out by using a humidity analyser MA 30 Sartorius

Parameters: mass of the sample=3 9, Temperature=100° C., Desiccation time=15 min

Result: Relative humidity=5.98%

Thickness Measurement

Procedure

Film manufacturing: 200 mg±20 mg of powder are deposited on a glass coverslip (dimension 50×25 mm) with the aid of a screen of 500 μm in such a way that a regular thin film is obtained. A gelose based on agar and artificial saliva (1.5/98.5 w/w) contained in a Petri dish is applied with a light pression on the glass coverslip; an instantaneous hydration of the powder and a film formation are occurring. After 1 min of hydration, the film is removed and the thickness is monitored with a thickness controller Braive Instrument®.

Results: 5 measurements are made at various points of the film on 3 different films. Mean thickness=252.6 μm

Tack Measurement

This test is similar to the so-called “Probe Tack” one—ASTM standard D 2979, it is carried out by using a traction equipment.

Procedure: about 30 mg of powder are deposited on porcine mucous membrane having a surface of 180 mm² previously moistened with artificial saliva and fixed on a glass plate. Another mucous membrane of an equivalent surface previously moistened with artificial saliva is fixed on a needle which compress the film-forming powder positioned below the equipment, and through the use of a force sensing device, the peel strength from the mucous membrane of the formed film (Cf. Schema below) is measured.

Parameter:

Compression speed=50 mm/min
Compression force 20N
Time for maintaining the force=1 min
Traction speed=50 mm/min
   Result:

Mean force of tack=3.3N (∂=0.8)

Resistance to Liquid Tests—Measurement of the Contact Angle Formed by a Liquid at the Time t

Procedure:

Film manufacturing: 200 mg±20 mg of powder are deposited on a glass coverslip (dimension 50×25 mm) with the aid of a screen of 500 μm, in such a way that a regular thin film is obtained. A gelose based on agar and artificial saliva (1.5/98.5.w/w) contained in a Petri dish is applied with a light pression on the glass coverslip; an instantaneous hydration of the powder and a film formation are occurring.

Sample cutting: with a scalpel, a gelose/film cut is carried out in order to perform the measurement.

Measuring device used: instrumented Goniometer KRUSS G2

Effected measurements at t=15 s, t=30 s, t=60 s

Number of measurements effected: 10

Tested liquids: Mineral water at 22° C., Coca-Cola at 22° C., Sodium bicarbonate solution at 22° C., Mineral water at 40° C.

Results:

TABLE 6

| Tested Liquid | t = 15 s | t = 30 s | t = 60 s |
|---|---|---|---|
| Mineral water 22° C. | 104° | 93° | 82° |
| Mineral water 40° C. | 76° | 68° | 54° |
| Coca-cola 22° C. | 103° | 91° | 81° |
| Sodium bicarbonate 22° C. | 89° | 73° | 63° |

A decrease of the contact angle in the course of the time is observed but there is no complete absorption of the liquid whatever the pH of the liquid (acid, basic or neutral), the film is semipermeable.

### EXAMPLE 3

### Micronized Film-Forming Powder According to the Invention

A powder having the composition by weight described in detail in the table 7 below is prepared.

TABLE 7

| Components | Quantity in % |
|---|---|
| 17 β-Estradiol | 5 |
| Hydroxypropyl cellulose | 53.2 |
| Carbopol 974P NF | 32.3 |
| Eudragit RSPO | 9.5 |

Manufacturing Process

The various components are mixed in a mixer-granulator of the mixer-granulator-fluidized air bed drier equipped with a top spray, nozzle or equivalent until the mixture is homogenized. Next, a wetting solution or suspension is sprayed with a spraying nozzle on the moving product in order to simultaneously distribute homogeneously the solution and to dry it for evaporating the granulation solvent.

This granulate is sized and then micronized with a air jet micronization device of GALETTE ALPINE 200 AS type, according to the following parameters:

Injector 7 bars; Crown: 6 Bars; Speed: 25 kg/h.

Final Product Controls

Particle size: carried out using a laser granulometer MASTER SIZER 2000 equipped with a vibrator Scirocco 2000.

Result: mean particle size before micronization=118.581 μm; after micronization=10.610 μm.

Thickness Measurement:

Procedure:

identical to the thickness measurement of the example 2

Results: 5 measurements are made at various points of the film on 3 different films. Mean Thickness=254.9 μm

Relative Humidity Level Measurement

Carried out by using a humidity analyser MA 30 Sartorius

Parameters: sample mass=3 g, Temperature=100° C., dessication time=15 min

Result Relative humidity 3.44%

Tack Measurement:

Procedure:

identical to the tack measurement of the example 2

Result

Mean force of tack 3.4N (∂=0,4)

Resistance to Liquid Tests—Measurement of the Contact Angle Formed by a liquid at the Time t

Procedure:

Identical to the resistance to liquid tests described in the example 2

Results: the results are represented in the table 8 below.

TABLE 8

| Tested Liquid | t = 15 s | t = 30 s | t = 60 s |
|---|---|---|---|
| Mineral water 22° C. | 58.5° | 45° | 37° |
| Mineral water 50° C. | 64° | 52° | 44° |
| Coca-cola 22° C. | 86° | 73° | 64° |
| Sodium bicarbonate 22° C. | 69° | 64° | 62° |

While the contact angle decreases significantly in the course of the time, the liquid is not totally absorbed by the adhesive, the film is semipermeable.

1. Micronized film-forming powder having a particle size of at most 100 μm and comprising the combination of at least an active substance, at least a biocompatible adhesive agent and at least a plasticizer.

2. Micronized film-forming powder according to claim 1, characterized in that the active substance is micronized.

3. Micronized film-forming powder according to claim 1, characterized in that it has a particle size of at most 50 μm.

4. Micronized film-forming powder according to claim 1, which has a particle size of at most 20 μm.

5. Micronized film-forming powder according to claim 1, which further comprises at least one compound selected from the group consisting of a surfactant, a wetting agent, a binder, a retardant, a penetration enhancer, a bioerodible diluent, a colorant, a flavor, a pH controlling agent and a combination of at least two of these compounds.

6. Micronized film-forming powder according to claim 1, wherein the active substance is a member selected from the group consisting of estradiol and its derivatives, norethisterone acetate, progesterone, testosterone, trinitrine, fentanyl, nitroglycerine, nicotine (S(−)-nicotine), scopolamine, clonidine, isosorbide dinitrate, levonorgestrel in combination with ethinylestradiol or with estradiol, androstanolone, alclometasone dipropionate, acetazolamide, acyclovir, adapalene, alclomethasone dipropionate, amcinonide, ameleine, bamethan sulphate+escin, betamethasone valerate, betamethasone dipropionate, bufexamac, caffeine, calcipotriol monohydrate, cetrimonium bromide, clobetasol propionate, crilanomer, desonide, dexpanthenol, diclofenac, diflucortolone, valerate, difluprednate, diphenydramine hydrochloride, econazole nitrate, erythromycin, flumetasone pivalate, fluocinolone acetonide, fluocinodine, fluocortolone, fluocortolone hexanoate, fluocortolone pivalate, hydrocortisone, hydrocortisone acetate, ibacitabin, ibuprofen, imiquimod, ketoconazole, ketoprofen, lidocaine, metronidazole, miconazole nitrate, minoxidil, niflumide acid, penciclovir, benzoyl peroxide, piroxam, iodinated povidone, promestriene, pyrazonibutasone, roxithromycin, sulphacetamide, triamconolone, tazarotene, tretinoin and isotretinoin, triclocarban, vidarabine monophosphate, β₃-adrenergic agonist, growth hormone, oxybutinin, buprenorphine, pergolide, estradiol+nestorone, nesterone, 7α-methyl-19-nortesterone, mecamylamine (antagonist of nicotine)+nicotine, salbutamol, selegiline, buspirone, ketotifen, lidocaine, testosterone+estradiol, ketorolac, eptazocine, insulin, α-interferon, prostaglandines, 17β-estradiol+norethindrone acetate, 5-aminolevulinic acid, benzodiazepine alprozolam, diclofenac, fenoprofen, flubiprofen, ketoprofen, methyl phenidate, miconazole, piroxicam, bruprenorphine, alprozolam, dexmedetomidine, prazosin (α-adrenergic antagonist), gestodene+ethinylestradiol, alprostadil, tulobuterol (β-adrenergic agonist), ethinylestradiol+norelgestromin, ketorolac, physostigmine, lidocaine, medindolol (α-adrenergic agonist), rotigotine (D2 dopamine antagonist), ethinylestradiol+norethindrone acetate, thiatolserine, and a combination of at least two thereof.

7. Micronized film-forming powder according to claim 1, wherein the active substance is a member selected from the group consisting of an emollient, a moisturizing agents, a vitamins, complex of fruit and an amino acid, and an antioxidant agents.

8. Micronized film-forming powder according to claim 1, wherein the active substance is a member selected from the group consisting of a vitamins, an inorganic salt, and brewer's yeast.

9. Micronized film-forming powder according to claim 1, wherein the bioadhesive agent is a member selected from the group consisting of ethyl cellulose, methyl cellulose, carboxymethyl cellulose, carboxymethyl cellulose sodium, hydroxyethyl cellulose, hydroxypropyl cellulose, hydroxypropyl methyl cellulose sodium, polyvinylpyrrolidone, a polyvinyl alcohol, polyisobutylene, polyisopropene, xanthan gum, locust bean gum, chitosan, chitosan chloride, a polycarboxylat, a carbomers acrylic/methacrylic acid copolymer, acrylic acid/acrylamide copolymer, acrylic acid/methyl methacrylate copolymer, acrylic acid/polyethylene glycol copolymer, polyacrylic acid/butyl acrylate copolymer, HEMA (2-hydroxyethyl methacrylate) copolymerized with Polymeg® (polytetramethylene glycol), Cydot® marketed by 3M (carbopol combined with polyisobutylene), pectin (of low viscosity), polyethylene oxide, methyl vinyl ether/maleic anhydride copolymer, tragacanth, monomethyl ether, monomethacrylate, drum dried waxy maize starch, sodium stearyl fumarate, sodium hyaluronate, guar gum, sodium alginate, a starches, dextran and a mixture& thereof.

10. Micronized film-forming powder according to claim 1, wherein the plasticizer is a member selected from the group consisting of dibutyl phthalate, dibutyl sebacate, acetyltributyl citrate, acetyltriethyl citrate, tributyl citrate, tributylethyl citrate, triacetin, PEG, propylene glycol, glycerol, a glycerol monoester and a derivatives thereof, castor oil and a mixtures thereof.

11. Micronized film-forming powder according to claim 1, wherein the surfactant is at least one nonionic surfactants.

12. Micronized film-forming powder according to claim 1, wherein the wetting agent comprises at least one polyol.

13. Micronized film-forming powder according to claim 1, wherein the binder is a member selected from the group consisting of acacia, alginic acid, carboxymethyl cellulose sodium, microcrystalline cellulose, a dextrin, ethyl cellulose, gelatin, glucose, guar gum, hydroxypropyl methyl cellulose, methyl cellulose, polyethylene oxide, povidone, pregelatinized starch and a mixtures thereof.

14. Micronized film-forming powder according to claim 1, wherein the retardant is a member selected from the group consisting of hydroxypropyl methyl cellulose acetate or succinate, hydroxypropyl methyl cellulose, hydroxypropyl cellulose, ethyl cellulose, hydroxyethyl cellulose, carboxymethyl cellulose sodium, a polyvinyl alcohol, a hydrocolloid an alginate, guar gum, xanthan gum, gum Arabic, agar, dextrin, carragheenan, polyethylene oxide, a carbomer, a polymer and or copolymer of acrylic acid, of methyl methacrylate, of polyvinyl acetate, or of carboxymethyl acetate, and a mixtures thereof.

15. Micronized film-forming powder according to claim 1, wherein the bioerodible diluent is a member selected from the group consisting of calcium or sodium carbonate or bicarbonate, sucrose, mannitol, xylitol, sorbitol, lactose, cellulose or microcrystalline cellulose powder, starch or a derivatives thereof, dibasic calcium phosphate, tribasic calcium phosphate, calcium sulphate, a dextrat, a dextrin, a dextrose excipient, fructose, kaolin, lactitol and a mixtures thereof.

16. Micronized film-forming powder according to claim 1, wherein the penetration enhancer is a member selected from the group consisting of an aliphatic fatty acid esters a fatty acid, an alcohol or polyol, an alpha-hydroxy acids and a mixture thereof.

17. Pharmaceutical, cosmetic or nutraceutical composition comprising a micronized film-forming powder according to claim 1 forming a film after application in situ on a hydrated support.

18. A method which comprises administering a composition according to claim 17, by the mucosal route.

19. A method which comprises administering a composition according to claim 17, on buccal mucosa, nasal mucosa or vaginal mucosa.

20. A method which comprises administering a composition according to claim 17, by the transdermal route with local or systemic effect.

21. Composition according to claim 17, characterized in that it is in pulverizable form.

22. Micronized film-forming powder according to claim 9 wherein the carbomer is carbopol.

23. Micronized film-forming powder according to claim 11 wherein the nonionic surfactant comprises at least one member selected from the group consisting of polyoxyethylene sorbitan (fatty acid ester), polyoxyethylene alkyl ether, and polyoxyethylene derived from castor oil.

24. Micronized film-forming powder according to claim 12 wherein the polyol comprises at least one member selected from the group consisting of sorbitol, glycerin, and polyethyleneglycol.

25. Micronized film-forming powder according to claim 14 wherein the hydrocolloid is a pectin.

26. Micronized film-forming powder according to claim 16 wherein the aliphatic fatty acid ester is isopropyl myristate; the fatty acid is oleic acid; and the alcohol or polyol is ethanol, propylene glycol or polyethylene glycol.
